(19) United States
(12) Patent Application Publication
(10) Pub. No.: US 2024/0325214 A1
(43) Pub. Date: Oct. 3, 2024

(54) PANT-TYPE WEARABLE ARTICLE

(71) Applicant: The Procter & Gamble Company, Cincinnati, OH (US)

(72) Inventors: Wei WU, Beijing (CN); Gueltekin ERDEM, Cincinnati, OH (US); Chunmin CHENG, Cincinnati, OH (US); Koichi MORIMOTO, Beijing (CN); Yi YUAN, Beijing (CN); Meng CHEN, Beijing (CN); Xiuyan TANG, Beijing (CN)

(21) Appl. No.: 18/738,208

(22) Filed: Jun. 10, 2024

Related U.S. Application Data
(63) Continuation of application No. PCT/CN2022/077996, filed on Feb. 25, 2022.

(30) Foreign Application Priority Data
Dec. 20, 2021 (WO) PCT/CN2021/139618

(51) Int. Cl. A61F 13/49; A61F 13/15; A61F 13/496; A61F 13/51; A61F 13/514; A61F 13/53

(52) U.S. Cl. CPC A61F 13/49011; A61F 13/15203; A61F 13/496; A61F 13/51478; A61F 2013/15292; A61F 2013/51052; A61F 2013/51452; A61F 2013/530481

(57) ABSTRACT

Disclosed is a wearable article continuous in a longitudinal direction and a transverse direction comprising front and back elastic belt regions, a crotch region, and a pair of side seams which join the front elastic belt region and the back elastic belt region to form a waist opening and a pair of leg openings. The crotch region extends longitudinally between the front elastic belt region and the back elastic belt region. Eac of the front and back elastic belt region comprises a laminate comprising an inner sheet, an outer sheet, and a plurality of elastic members running in the transverse direction. The outer sheet comprises a garment facing surface and a wearer facing surface. The outer sheet is formed by a multi-fiber layer nonwoven having a basis weight of from about 16gsm to about 35gsm and comprises a garment facing layer and a wearer facing layer.

### CROSS REFERENCE TO RELATED APPLICATIONS

This application claims the benefit, under 35 U.S.C. § 120, of Application No. PCT/CN2022/077996, filed on Feb. 25, 2022, which claims foreign priority, under 35 U.S.C. § 119, of Application No. PCT/CN2021/139618, filed on Dec. 20, 2021, both of which are fully incorporated by reference herein.

### FIELD

The present invention relates to pant-type wearable articles having an elastic belt with improved softness. The present invention also relates to wearable articles having improved softness in the crotch region.

### BACKGROUND

Infants and other incontinent individuals wear absorbent articles such as diapers to receive and contain urine and other body exudates. Pull-on absorbent articles, or pant-type absorbent articles, are those which are donned by inserting the wearer's legs into the leg openings and sliding the article up into position about the lower torso. Pant-type absorbent articles have become popular for use on children who are able to walk and often who are toilet training, as well as for younger children who become more active in movement such that application of taped-type absorbent articles tends to be more difficult, and also for younger babies requiring a soft fit around the waist opening and leg openings.

Pant-type articles may take various structures wherein the circumference of the waist opening and vicinity thereof is made elastic enough to facilitate the wearer or the caregiver to expand the article and insert the wearer's legs into the leg openings for wearing the article. The region of the waist circumference and vicinity thereof is often referred to as the elastic belt. One type of structure for the pant-type article is the belt-type pant having a central chassis to cover the crotch region of the wearer and a separate elastic belt defining the waist opening and leg opening, such as described in PCT Publication WO 2006/17718A. Another type of structure for the pant-type article is the uni-body pant configured such that the outer cover of the article completely covers the entirety of the garment-facing surface of the article, wherein the portion configured to stretch about the torso is considered the elastic belt region.

Whatever the structure of the pant-type article may be, pant-type articles provide only a very small range of size adjustment or body configuration adjustment based on the structural limitations of the article. As such, pant-type articles are typically so configured to accommodate size and configuration ranges by providing the elastic belt region very stretchable and comfortable to wear, yet with reliable fit such that sufficient protection against sagging and leakage may be provided. Further, the elastic belt region may be the portion which is most touched and observed by the wearer or the caregiver upon use, and thus its properties most associated with the function and quality of the article. An elastic belt having soft tactile sense may connote high quality and function of the article, which is advantageous.

Based on the foregoing, there is a need for a wearable article providing improved softness, while maintaining stretchability for ease of application, fit for preventing sagging, and comfort and improved breathability for skin health. There is also a need for providing such a wearable article which can be economically made.

### SUMMARY

The present invention is directed to a wearable article continuous in a longitudinal direction and a transverse direction comprising a front elastic belt region, a back elastic belt region, a crotch region, and a pair of side seams which join the front elastic belt region and the back elastic belt region to form a waist opening and a pair of leg openings; the crotch region extending longitudinally between the front elastic belt region and the back elastic belt region;
wherein each of the front and back elastic belt region comprises a laminate comprising an inner sheet, an outer sheet, and a plurality of elastic members running in the transverse direction;
wherein the outer sheet comprises a garment facing surface and a wearer facing surface, the outer sheet formed by a multi-fiber layer nonwoven having a basis weight of from about 16 gsm to about 35 gsm and comprising a garment facing layer and a wearer facing layer, the garment facing layer comprising fibers having a diameter of about 11 μm or less, preferably from about 7 μm to about 11 μm, and the wearer facing layer comprising fibers having a diameter of about 13 μm or more, preferably from about 13 μm to about 24 μm, wherein the weight ratio of the garment facing layer is from about 20% to about 70% of the multi-fiber layer nonwoven.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings and which like designations are used to designate substantially identical elements, and in which:

FIG. 1A is a perspective view of one embodiment of a wearable article of the present invention.

FIG. 1B is a schematic view of one embodiment of a wearable article of the present invention showing the front side of the article.

FIG. 2 is a schematic plan view of one embodiment of a wearable article of the present invention with the seams unjoined and in a flat uncontracted condition showing the garment facing surface.

FIGS. 3A-3C are SEM images of a cross-sectional view of a multi-fiber layer nonwoven of the present invention.

FIG. 4 is a schematic view of an example of a hanger-type sample holding fixture according to the “Whole Article Force Measurement”.

FIG. 5 is a schematic cross section view of an embodiment of the central chassis of the present invention with the thickness (Z direction) exaggerated.

FIG. 6 is a schematic plan view of one embodiment of a laminate of the present invention showing the elastic member positioning, elastic adhesive bondings and area where patterns of discrete bond units are disposed.

### DEFINITIONS

As used herein, the following terms shall have the meaning specified thereafter:

“Wearable article” refers to articles of wear which may be in the form of pants, taped diapers, incontinent briefs, feminine hygiene garments, and the like. The “wearable article” may be so configured to also absorb and contain various exudates such as urine, feces, and menses discharged from the body. The “wearable article” may serve as an outer cover adaptable to be joined with a separable disposable absorbent insert for providing absorbent and containment function, such as those disclosed in PCT publication WO 2011/087503A.

“Pant” refers to disposable wearable articles having a pre-formed waist and leg openings. A pant may be donned by inserting a wearer's legs into the leg openings and sliding the pant into position about the wearer's lower torso. Pants are also commonly referred to as “closed diapers”, “prefastened diapers”, “pull-on diapers”, “training pants” and “diaper-pants”.

“Longitudinal” refers to a direction running substantially perpendicular from a waist edge to an opposing waist edge of the article and generally parallel to the maximum linear dimension of the article.

“Transverse” refers to a direction perpendicular to the longitudinal direction.

“Proximal” and “distal” refer respectively to the position closer or farther relative to the longitudinal center of the article.

“Wearer facing” and “garment-facing” refer respectively to the relative location of an element or a surface of an element or group of elements. “Wearer facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable wearable article).

“Disposed” refers to an element being located in a particular place or position.

“Joined” refers to configurations whereby an element is directly secured to another element by affixing the element directly to the other element and to configurations whereby an element is indirectly secured to another element by affixing the element to intermediate member(s) which in turn are affixed to the other element.

“Film” refers to a sheet-like material wherein the length and width of the material far exceed the thickness of the material. Typically, films have a thickness of about 0.5 mm or less.

“Water-permeable” and “water-impermeable” refer to the penetrability of materials in the context of the intended usage of disposable wearable articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable”.

“Extendibility” and “extensible” mean that the width or length of the component in a relaxed state can be extended or increased.

“Elasticated” and “elasticized” mean that a component comprises at least a portion made of elastic material.

“Elongatable material”, “extensible material”, or “stretchable material” are used interchangeably and refer to a material that, upon application of a biasing force, can stretch to an elongated length of at least about 110% of its relaxed, original length (i.e. can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

“Dimension”, “Length”, “Width”, “Pitch”, “Diameter”, “Aspect Ratio”, “Angle”, and “Area” of the article are all measured in a state wherein the article is extended to the Full Stretch Circumference W1 according to the “Whole Article Force Measurement” herein, and utilizing a ruler or a loupe, unless specified otherwise.

“Basis weight” of a nonwoven substrate or other material is measured by EDANA method 20.2-89.

“Artwork” refers to a visual presentation to the naked eye, which is provided by printing or otherwise, and having a color. Printing includes various methods and apparatus well known to those skilled in the art such as lithographic, screen printing, flexographic, and gravure ink jet printing techniques.

“Color” or “Colored” as referred to herein includes any primary color except color white, i.e., black, red, blue, violet, orange, yellow, green, and indigo as well as any declination thereof or mixture thereof. The color white is defined as those colors having a L* value of at least 94, an a* value equal to 0±2, and a b* value equal to 0±2 according to the CIE L* a* b* color system.

### DETAILED DESCRIPTION

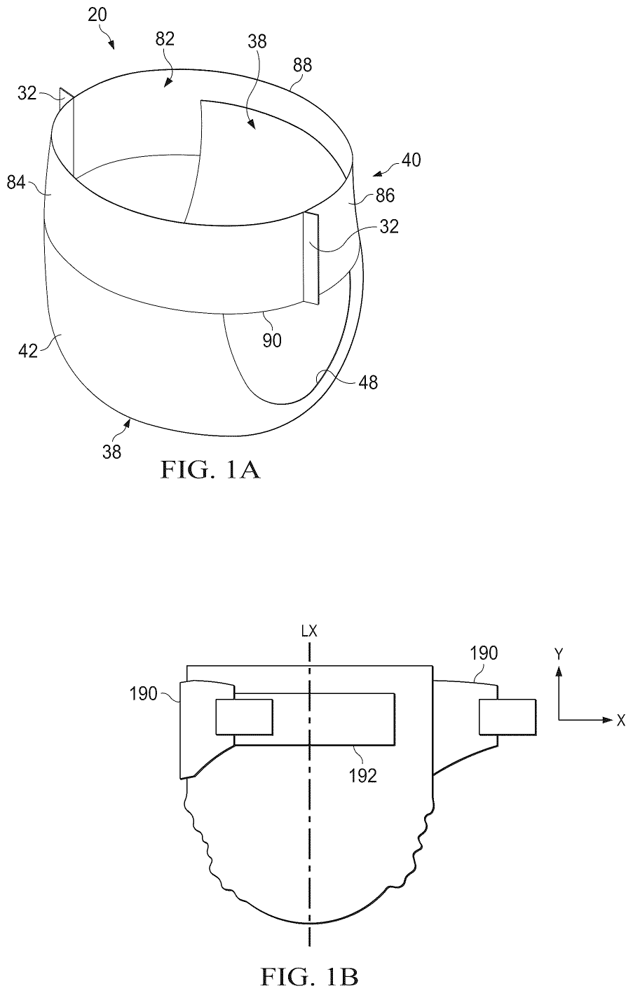

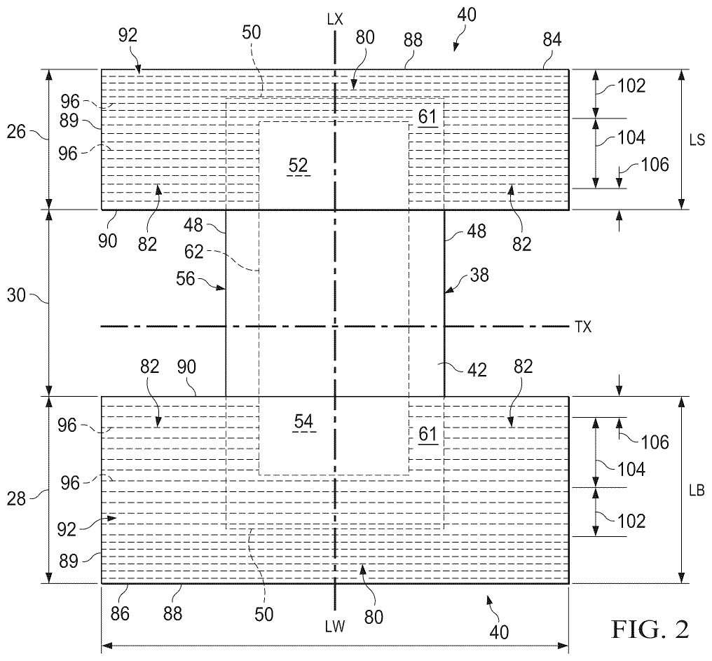

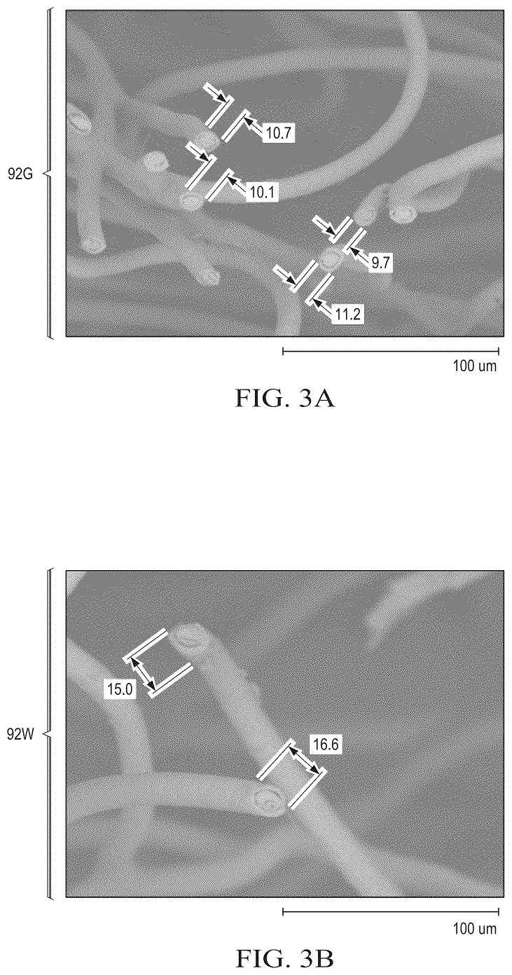

Wearable Article FIG. 1A is a perspective view of a wearable article (20) of the present invention, and FIG. 2 is a schematic plan view of a wearable article with the seams unjoined and in its flat uncontracted condition showing the garment-facing surface. FIG. 1B is a schematic perspective view of another type of wearable article. The wearable article (20) has a longitudinal centerline LX which also serves as the longitudinal axis, and a transverse centerline TX which also serves as the transverse axis. The wearable article (20) has a body facing surface, a garment facing surface, a front elastic belt region (84), a back elastic belt region (86), a crotch region (30), and side seams (32) which join the front elastic belt region (84) and the back elastic belt region (86), to form two leg openings and a waist opening.

The wearable article (20) may be a belt-type pant as in FIGS. 1A and 2 comprising a central chassis (38) to cover the crotch region (30) of the wearer, a front elastic belt (84) and a back elastic belt (86) (hereinafter may be referred to as “front and back elastic belts”), the front and back elastic belts (84, 86) forming a discrete ring-like elastic belt (40) extending transversely defining the waist opening. For the belt-type pant, the discrete ring-like elastic belt (40) may also be referred to as the elastic belt (40). For the belt-type pant as in FIGS. 1A and 2, the front and back elastic belts (84, 86) and the central chassis (38) jointly define the leg openings. For the belt-type pant, the front elastic belt (84) is the front region (26), and the back elastic belt (86) is the back region (28), and the remainder is the crotch region (30). While not shown, the wearable article (20) may be a uni-body type pant configured such that the outer cover of the central chassis (38) and the elastic belt (40) are common. For the uni-body type pant, the portion extending in the transverse direction between the side seams (32), respectively, are considered the front region (26) and the back region (28), and the remainder is the crotch region (30). For the uni-body type pant, the front region (26) is considered the front elastic belt region (84), and the back region (28) is considered the back elastic belt region (86).

The central chassis (38) may comprise a liquid permeable topsheet (24), a liquid impermeable backsheet (25) and an absorbent core (62) disposed between the topsheet (24) and the backsheet (25), and further an outer cover layer (42) for covering the garment-facing side of the backsheet (25). The outer cover layer (42) may be a nonwoven sheet. The central chassis (38) may contain an absorbent core (62) for absorbing and containing body exudates disposed on the central chassis (38), and an absorbent material non-existing region (61) surrounding the periphery of the absorbent core (62). The absorbent material non-existing region (61) may be made of the topsheet (24) and/or the backsheet (25) and/or the outer cover layer (42) and/or other parts configuring the central chassis (38). In the embodiment shown in FIG. 2, the central chassis (38) has a generally rectangular shape, left and right longitudinally extending side edges (48) and front and back transversely extending end edges (50). The absorbent core (62) may exist through the entire longitudinal dimension of the crotch region and extending at least partly in the front region (26); or at least partly in both the front and back regions (26, 28). The central chassis (38) may have a front waist panel (52) positioned in the front region (26) of the wearable article (20), a back waist panel (54) positioned in the back region (28), and a crotch panel (56) between the front and back waist panels (52, 54) in the crotch region (30). The center of the front elastic belt (84) is joined to a front waist panel (52) of the central chassis (38), the center of the back elastic belt (86) is joined to a back waist panel (54) of the central chassis (38), the front and back elastic belts (84, 86) each having a left side panel and a right side panel (82) where the central chassis (38) does not overlap. The central chassis has a crotch panel (56) positioned between the front waist panel (52) and the back waist panel (54).

### Elastic Belt

The elastic belt (40) of the article of the present invention acts to dynamically create fitment forces and to distribute the forces dynamically generated during wear. The front and back elastic belts (84, 86) may be joined with each other only at the side edges (89) to form side seams (32), a waist opening and two leg openings. Each leg opening may be provided with elasticity around the perimeter of the leg opening. The elasticity around the leg opening may be provided by the combination of elasticity from the front belt (84), the back belt (86), and the central chassis (38).

The front elastic belt (84) and back elastic belt (86) are configured to impart elasticity to the belt (40). Referring to FIGS. 1A and 2, the front belt (84) and the back belt (86) may each comprise a laminate, the laminate comprising an outer sheet (92), an inner sheet (94), and a plurality of elastic members (96) running in the transverse direction. The elastic belt region (40) may be closely associated with the function and quality of the article. Thus, materials for forming the elastic belt region (40), as well as the gathering profile of the elastic belt region, are carefully selected by the manufacturer to provide the desired tactile and visible senses. Tactile sense such as flexibility and cushiony touch may enhance perception of high quality. For providing such favorable tactile sense, the outer sheet (92) of the present invention comprises a garment facing surface and a wearer facing surface, the outer sheet (92) formed by a multi-fiber layer nonwoven (MLN) having a basis weight of from about 16 gsm to about 35 gsm and comprising a garment facing layer (92G) and a wearer facing layer (92W), the garment facing layer (92G) comprising fibers having a diameter of about 11 μm or less, preferably from about 7 μm to about 11 μm, and the wearer facing layer (92W) comprising fibers having a diameter of about 13 μm or more, preferably from about 13 μm to about 24 μm, wherein the weight ratio of the garment facing layer (92G) is from about 20% to about 70% of the multi-fiber layer nonwoven (MLN).

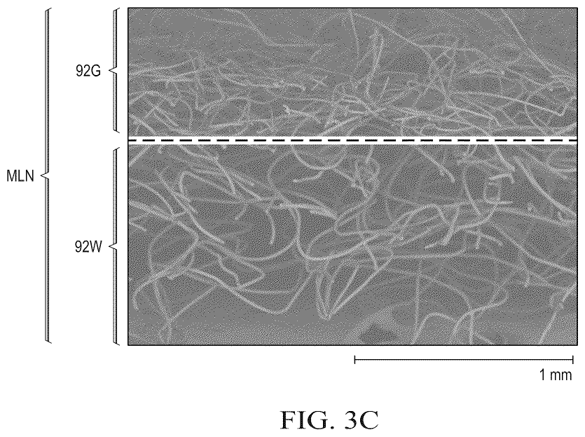

By multi-fiber layer nonwoven (MLN) herein, what is meant is a nonwoven comprising distinct layers in the thickness direction of fibers of different diameter size. Referring to FIG. 3A, the multi-fiber layer nonwoven (MLN) may be made of 2 layers, namely the garment facing layer (92G) and the wearer facing layer (92W), or may be made of more than 2 layers, having an additional layer between the garment facing layer (92G) and the wearer facing layer (92W). The garment facing layer (92G) comprises fibers having a diameter of about 11 μm or less, preferably from about 7 μm to about 11 μm, as measured according to the method herein. Fibers of such fineness are believed to provide a very smooth tactile sense when touched, as the fibers are below distinctive perceivable distance of tactile sensory to the human skin. The wearer facing layer (92W) comprising fibers having a diameter of about 13 μm or more, preferably from about 13 μm to about 24 μm, as measured according to the method herein. The fibers of the wearer facing layer (92W) provide some structural strength and cushiony tactile sense. Without being bound by theory, by providing the multi-fiber layer nonwoven (MLN) to have a basis weight of from about 16 gsm to about 35 gsm and a weight ratio of the garment facing layer (92G) of from about 20% to about 70%, a nonwoven material having balanced softness attributes such as smoothness, cushiony feel, and lack of grains/neps/lumps feeling is provided. The measurement for obtaining the diameter of the fibers and the weight ratio of the garment facing layer are provided in further detail below.

The multi-fiber layer nonwoven (MLN) for forming the outer sheet (92) may be made by processes such as spunbond, spunlace, carded or air-laid; and may comprise fibers and/or filaments made of polypropylene (PP), polyethylene (PE), polyethylene phthalate (PET), polylactic acid/polylactide (PLA) or conjugate fibers (such as PE/PET, PE/PP, PE/PLA) as well as natural fibers such as cotton or regenerated cellulosic fibers such as viscose or lyocell. The outer sheet (92) nonwoven may be a multilayer or composite structure combining nonwovens made by different processes and fibers such as combining spunbond and carded nonwovens. The outer sheet (92) nonwoven may be made by biodegradable material, or derived from renewable resources. Exemplary material for the outer sheet (92) include: air-through carded nonwoven having a thickness of at least about 50 μm, or at least about 80 μm, or at least about 200 μm. Such material may provide a soft lofty feeling to the garment-facing side. Suitable for the outer sheet (92) nonwoven of the present invention are air-through carded nonwoven material made of co-centric bicomponent fiber, crimping fiber made through core eccentric bicomponent filament or side by side bicomponent filament. One non-limiting material for the multi-fiber layer nonwoven (MLN) is a bicomponent fiber made of PE sheath and PET core which is airlaid. When such material is utilized, the fibers of the garment facing layer (92G) may be from about 0.6 to about 0.8 denier, and the fibers of the wearer facing layer (92W) may be from about 1.0 to about 2.0 denier. Non-limiting examples of commercially available materials suitable for the outer sheet (92) nonwoven of the present invention include: 16-35 gsm air-through carded nonwoven substrate comprising PE/PET bi-component fibers, such as those available from Jiangsu Wisdom Nonwoven Co. Ltd. or Xiamen Yanjan New Material Co. Ltd.

The inner sheet (94) of the present invention may be a nonwoven having a basis weight of from about 5 gsm to about 45 gsm, or from about 5 gsm to about 35 gsm. The inner sheet (94) nonwoven may have a fiber diameter of from about 0.5 dpf to about 4 dpf. The inner sheet (94) nonwoven may be made by processes such as spunbond, spunlace, carded or air-laid; and may comprise fibers and/or filaments made of polypropylene (PP), polyethylene (PE), polyethylene phthalate (PET), polylactic acid/polylactide (PLA) or conjugate fibers (such as PE/PET, PE/PP, PE/PLA) as well as natural fibers such as cotton or regenerated cellulosic fibers such as viscose or lyocell. The inner sheet (94) nonwoven may also be a multilayer or composite structure combining nonwovens made by different processes and fibers such as combining spunbond and carded nonwovens. The inner sheet (94) nonwoven may be made by biodegradable material, or derived from renewable resources. Non-limiting examples of materials suitable for the inner sheet (94) nonwoven of the present invention include: 8-45 gsm spun melt nonwoven substrate comprising PP monofilament or PE/PP bi-component fibers, such as those available from Malaysia Fibertex, Avogl China, 12-30 gsm air-through carded nonwoven substrate made of PE/PET bi-component staple fiber, such as those available from Beijing Dayuan Nonwoven Fabric Co. Ltd. or Xiamen Yanjan New Material Co. Ltd., and 8-30 gsm spun melt nonwoven substrate comprising PP monofilament or PE/PP bi-component fibers, such as those available from Fibertex or Fitesa.

The basis weight of the outer sheet (92) and the inner sheet (94) may be adjusted such that the basis weight of the inner sheet (94) is not greater than the basis weight of the outer sheet (92). Thus, the outer sheet (92) may be provided with a soft lofty tactile sense which connotes high quality, while the inner sheet (94) may be kept thinner and conforming to the outer sheet (92), thus saving cost. Further, without being bound by theory, by providing the basis weight relationship as such, it is believed that skin sweating is effectively transported to the outer sheet (92) and outside the laminate, while preventing the transported sweat back to the inner sheet (94). The hydrophilicity/hydrophobicity of the outer sheet (92) and the inner sheet (94) is adjusted such that the hydrophilicity of the outer sheet (92) is higher than that of the inner sheet (94). Without being bound by theory, it is believed that such gradient of hydrophilicity is advantageous in transporting skin sweat from the inner sheet (94) to the outer sheet (92) and outside the laminate. The inner sheet (94) nonwoven may be inherently hydrophobic. The inner sheet (94) nonwoven may be provided hydrophobicity by treating with hydrophobic melt additives into polymer resin in the fiber making process, or by applying hydrophobic additives after the nonwoven is formed. The outer sheet (92) nonwoven may inherently be hydrophobic, and thus provided relatively more hydrophilic than the inner sheet (94) by treating with hydrophilic melt additives into polymer resin in the fiber making process, or by applying hydrophilic additive after the nonwoven is formed.

Referring to FIG. 2, the elastic member (96) may be made by a plurality of elastic strands (96) running parallel to each other in the transverse direction, wherein the laminate has a region wherein the elastic strands (96) have a longitudinal pitch of from about 3 mm to about 18 mm, or from about 3 mm to about 12 mm, or from about 3 mm to about 7 mm.

The tensile stress (N/m) of the entirety of the front and back elastic belts (84, 86), respectively, may be profiled in order to provide the functional benefits of the present invention, such as ease of stretch and application, while also maintaining certain force during wear, to prevent the article from sagging after loading. When the elasticity of the front and back elastic belts (84, 86) are provided by a plurality of elastic members (96) running in the transverse direction, the tensile stress may be adjusted by one or more of the following methods; 1) elongation rate of the elastic member (96); 2) density (dtex) of the elastic member (96); 3) longitudinal pitch of multiple elastic members (96); and 4) effective length of elasticity of the elastic member (96) in the transverse direction. By elongation, “0% elongation” is meant the original length of the elastic member. When a portion of an elastic member (96) is removed of its elasticity, the remainder of the intact elastic member capable of imparting elasticity is defined as the “effective length of elasticity of an elastic member”.

The tensile stress profile of the elastic members may be so adjusted, such that the wearable article has a certain Stretch Circumference Force and Fit Circumference Force, according to the measurements herein. What is meant by Stretch Circumference Force is the loading force at a certain stretch level, which is believed to simulate initial stretch experience felt by the wearer or caregiver when inserting hands and stretch opening the article. Further, despite such relatively low Stretch Circumference Force, the elastic belt (40) of the present invention may maintain a suitable Fit Circumference Force, according to the measurements herein. What is meant by Fit Circumference Force is the unloading force at a certain stretch level, which is believed to simulate the force felt by the wearer while wearing the article. Accordingly, the article of the present invention has a Stretch Circumference Force of no more than about 6.5N, and a Fit Circumference Force of at least about 2.0 N according to the measurements herein. Without being bound by theory, it is believed that, by forming the outer sheet (92) by the multi-fiber layer nonwoven (MLN) as described above and providing the article with the aforementioned Stretch Circumference Force and Fit Circumference Force, the laminate is provided with an overall soft and lofty tactile sense as well as ease of application and comfortable fit, thus providing an overall favorable experience for the wearer as well as the caregiver.

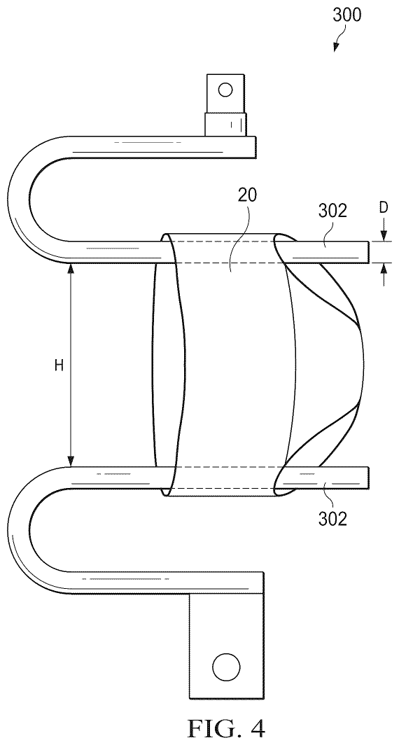

The measurements for obtaining the Stretch Circumference Force and Fit Circumference Force are provided in further detail below, with reference to FIG. 4.

Use of the multi-fiber layer nonwoven (MLN) for forming the outer sheet (92) is further advantageous as having a relatively high Material Breaking Point, according to the measurements herein. This is believed to be due to relatively more bonding points provided to the nonwoven by utilizing fine fibers. The Material Breaking Point of the multi-fiber layer nonwoven (MLN) herein may be at least about 7N, or from about 7N to about ION. Namely, due to the force applied to the side seam to tear open the side seam (32) in the lateral direction of the article, the substrate may rip in this direction. The article of the present invention has selected side seam properties to avoid such ripping, even when an inner sheet (94) or outer sheet (92) of relatively low Material Breaking Point are used. When the outer sheet (92) has a certain Material Breaking Point, this is believed to prevent the material to ripping in an undesired direction when tear opening the side seam by hand along the longitudinal dimension for removal from the wearer.

The inner sheet (94) for forming the laminate may be a nonwoven made of material having a melting point of no more than about 165° C. By providing such inner sheet (94), the laminate formed together with the aforementioned outer sheet (92) may provide side seams (32) which tolerate normal usage conditions, while also being easy to open after use for removal.

The side seam of the present invention has a Belt Minimum Peel Strength of at least about 6N/25 mm, or at least about 8N/25 mm, and a Belt Maximum Peel Strength of no more than about 18N/25 mm, according to measurements herein. What is meant by Belt Minimum/Maximum Peel Strength is the average minimum/maximum peel strength among the 4 portions of the seam over a certain number of ring-like elastic belts. Specifically, the strength of a side seam may be represented by 4 (four) unique parts of the 2 (two) seams per ring-like elastic belt, namely the distal (top) edges and proximal (bottom) edges of the opposed longitudinal edges of the left and right seams. These four unique parts may be identified as “top left”, “top right”, “bottom left”, and “bottom right”, and their forces identified as codes FTL, FTR, FBL, and FBR. An average peel strength of each of the four unique parts may be obtained over a certain number of ring-like elastic belts. What is meant by Belt Minimum Peel Strength is the lowest peel strength among the four unique parts. What is meant by Belt Maximum Peel Strength is the highest peel strength among the four unique parts. By controlling the four unique parts to have a seam strength within the required values over a certain number of ring-like elastic belts, a ring-like elastic belt having a seam strength that endures premature tearing during use while also being easy to open after use may be stably manufactured.

The side seam of the present invention has a Top Bottom Difference of no more than about 15%, or no more than about 13%, according to measurements herein. Referring to the peel strengths among the 4 portions of the seam described above, the Top Bottom Difference is obtained as the absolute value of difference between the top forces FTL and FTR compared to the bottom forces FBL and FBR:

$$\vert \{ ( FTL + FTR ) - ( FBL + FBR ) \} \div ( FBL + FBR ) \vert ( \% )$$

When the Top Bottom Difference is controlled to a small deviation, the peeling experience from the top to bottom of the seam is perceived smooth and easy.

The measurements for obtaining the Seam Maximum Peel Strength, the Seam Minimum Peel Strength, the Belt Maximum Peel Strength, the Belt Minimum Peel Strength, and Material Breaking Point, are provided in further detail below.

### Central Chassis

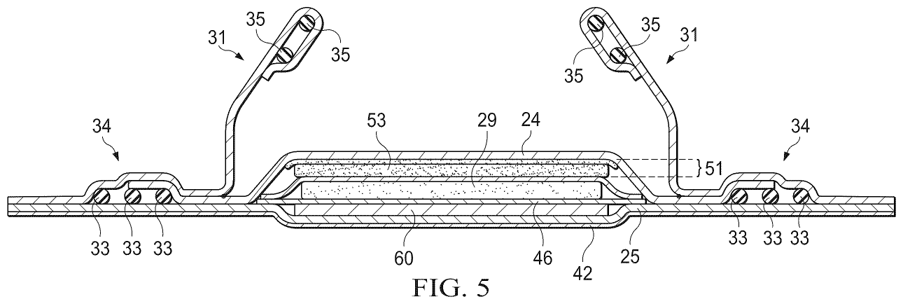

Referring to FIGS. 2 and 5, the central chassis (38) may comprise an absorbent core (62) for absorbing and containing body exudates disposed on the central chassis (38). The absorbent core (62) may include an absorbent layer and an acquisition system (51). The absorbent layer is the region wherein absorbent materials (29) having a high retention capacity, such as superabsorbent polymers, are present. The absorbent layer may be substantially cellulose free. Alternatively, the absorbent layer may contain cellulose. There may be an absorbent layer mainly comprising cellulose, and another absorbent layer mainly comprising superabsorbent polymers.

Superabsorbent polymers of the absorbent layer may be disposed between first and second layers of material immobilized by a fibrous layer of thermoplastic adhesive material. The first and second layers of materials may be nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process. The acquisition system (51) facilitates the acquisition and the distribution of body exudates and may be placed between the topsheet (24) and the absorbent layer. The acquisition system (51) may include cellulosic fibers.

The absorbent layers may be disposed in plurality in the absorbent core (62). Some portions of the absorbent layers may be configured to have substantially no absorbent material to form a channel or a plurality of channels. Channels may be useful for allowing the absorbent core (62) to bend upon swelling with fluids, such that the central chassis conforms to the wearer's body after swelling and prevent sagging of the article. The channels may also be formed in the acquisition system (51), and may be configured to at least partly match the channels of the absorbent layer in the thickness direction.

The absorbent core (62) may comprise a high loft material encompassing superabsorbent polymers. The term “high loft” refers to low density bulky fabrics, as compared to flat, paper-like fabrics. High loft webs are characterized by a relatively high porosity. This means that there is a relatively high amount of void space in which superabsorbent polymer particles can be distributed. The high loft material (without the superabsorbent particles) of the invention may have a density at a pressure of 4.14 kPa (0.6 psi) below 0.20 g/cm³, in particular ranging from 0.05 g/cm³ to 0.15 g/cm³. The high loft layer (without the superabsorbent particles) may have a density at a pressure of 2.07 kPa (0.3 psi) below 0.20 g/cm³, in particular ranging from 0.02 g/cm³ to 0.15 g/cm³. The high loft layer (without the superabsorbent particles) of the invention may have a density at a pressure of 0.83 kPa (0.12 psi) below 0.15 g/cm³, in particular ranging from 0.01 g/cm³ to 0.15 g/cm³, and a basis weight of from 15 to 500 gsm, preferably 30-200 gsm, such as those described in US 2021/0361497 A1. The absorbent core (62) comprising high loft material encompassing superabsorbent polymers may also contain channels.

Alternatively, the absorbent core (62) may comprise an absorbent layer having superabsorbent polymers disposed between first and second layers of nonwoven material immobilized by a fibrous layer of thermoplastic adhesive material (not shown). The first and second layers of nonwoven materials may be relatively low basis weight nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process. Such an embodiment is exemplarily shown in FIG. 5. In such embodiments, a) the intermediate layer (60) may be hydrophobic and the lower substrate layer (46) may be hydrophilic; or b) the intermediate layer (60) and the lower substrate layer (46) may both be hydrophilic and the intermediate layer (60) may be less hydrophilic than the lower substrate layer (46); or c) the intermediate layer (60) and the lower substrate layer (46) may both be hydrophobic and the lower substrate layer (46) may be less hydrophobic than the intermediate layer (60).

The absorbent core (62) may further comprise a liquid management layer (53) directly under the topsheet (24). The liquid management layer (53) may be part of the acquisition system (51). The function of such a layer is to rapidly acquire the fluid from the topsheet (24) away from the wearer-facing side and/or to distribute over a larger area so it is more efficiently absorbed by the absorbent core. It is also possible that such a liquid management layer (53) may be placed between the backsheet (25) and the absorbent core. The liquid management layer may be a spunlace nonwoven comprising viscose, PET, CoPET/PET fibers, and combinations thereof.

As described above, the central chassis (38) may comprise a nonwoven outer cover layer (42) for covering the garment-facing side of the backsheet (25). The outer cover layer (42) may be a nonwoven sheet. The outer cover layer (42) may be formed by the multi-fiber layer nonwoven suitable for forming the outer sheet (92). The outer cover layer (42) and outer sheet (92) may be formed by the same multi-fiber layer nonwoven material. By providing the entire garment-facing surface of the wearable article by the same multi-fiber layer nonwoven material, a wearable article having overall improved softness connoting high quality may be obtained.

### Intermediate Layer

The absorbent core (62) may comprise an intermediate layer (60) between the layer of absorbent material and the backsheet (25). The intermediate layer (60) may be in direct contact with the layer of absorbent material (29) and with the backsheet (25). The intermediate layer (60) may be useful as a masking layer to isolate the superabsorbent polymer particles in the layer of absorbent material from the backsheet (25), thus reducing graininess feeling and improving the tactile properties of the garment-facing side of the article, especially for absorbent core (62) containing a high level of superabsorbent polymer particles.

The intermediate layer (60) may further isolate the exudates which have been absorbed in the layer of absorbent material from the garment-facing side of the article, as this may be visually unpleasant to the caregiver. Thus by having an intermediate layer with a relatively high opacity, stains in the layer of absorbent material (e.g. from urine or feces) can be concealed from view, when looking at the backsheet (25) of the central chassis during use. The dry opacity of the intermediate layer may be at least 25%, or at least 40%, or at least 50%, or at least 70%. The intermediate layer (60) can also help reduce the residual moisture in contact with the backsheet (25), which may lead to cold/wet feeling for the caregiver, or may lead to the wearer mistaking the cold/wet feeling as liquid leaking out of the wearable article. The intermediate layer (60) may also serve as a temporary reservoir for liquid that had not been absorbed fast enough by the layer of absorbent material.

Additional layers provided to an absorbent core (62) generally increase the thickness and bulkiness of the article. This may lead to increased bending stiffness in the crotch region, thus acting as drawback for conformity and close contact of the article to the wearer's body, thereby reducing wearer comfort. Therefore, it is desirable for the intermediate material (60) to have a thickness that can survive compressive force, while also having a cushiony benefit even when compressed. Accordingly, the intermediate layer may have an MD Tensile/Basis Weight of no greater than about 0.75 N/5 cm/g/m² or greater than about 0.71 N/5 cm/g/m² as measured according to measurements herein, and a Thickness/Basis Weight of no less than about 0.078 mm/g/m², or no less than about 0.80 mm/g/m², or no less than about 0.90 mm/g/m² as according to measurements herein. Lower values for MD Tensile/Basis Weight indicate that a material is less bonded and more flexible versus higher values. Higher values for Thickness/Basis Weight indicate that a material is loftier under compression versus lower values.

The basis weight of the intermediate layer (60) may be homogeneous throughout longitudinal and transverse direction of the intermediate layer (60). The intermediate layer (60) may have a smaller extension in the longitudinal and/or transverse direction than the layer of absorbent material, such that absorbent material (29) extends beyond the intermediate layer in the longitudinal and/or transverse direction. Alternatively, the intermediate layer (60) may have a larger extension in the longitudinal and/or transverse direction than the absorbent material (29) when the absorbent layer is in direct contact with the intermediate layer.

The intermediate layer (60) may be a carded air-through bonded nonwoven, a carded calendar bonded nonwovens nonwoven web, a spunbond or meltblown nonwoven web (made of continuous fibers) or a nonwoven with spunbond and meltblown layers (e.g. an SMS, SMMS, SMSS or the like). In one embodiment, the intermediate layer (60) is a carded air-through bonded nonwoven. The nonwoven web may be made of synthetic fibers, such as polyolefin (e.g. polyethylene, polypropylene or mixtures or combinations thereof), polyethylene terephthalate (PET), co-PET, polylactic acid (PLA), polyhydroxy alkanoid (PHA), or combinations or mixtures thereof. The fibers may be continuous or staple fibers.

The intermediate layer (60) may comprise a nonwoven web comprising first thermoplastic fibers having a first melting temperature and second thermoplastic fibers having a second melting temperature, a difference of the first melting temperature and the second melting temperature is at least about 40° C., or at least 50° C., or at least 60° C. If melting temperature of different fiber types get close more or all fiber types will bond to each other and/or to itself which will result in excessive stiffness which is not desired. When the first thermoplastic fiber comprises at least two polymers having different melting temperatures, a melting temperature of a polymer lower than melting temperature(s) of any other polymer(s) constituting the first thermoplastic fiber is considered the first melting temperature. By the same token, when the second thermoplastic fiber comprises at least two polymers having different melting temperatures, one melting temperature of a polymer lower than melting temperature(s) of any other polymer(s) constituting the second thermoplastic fiber is considered the second melting temperature.

In the embodiment where the nonwoven web comprised by or forming the intermediate layer comprises first thermoplastic fibers having a first melting temperature and second thermoplastic fibers having a second melting temperature, a difference of the first melting temperature and the second melting temperature is at least about 40° C., the nonwoven web may comprise at least 40 wt %, or at least 50 wt %, or at least 60 wt % of the first or the second thermoplastic fibers whichever having a lower melting temperature based on the total weight of the nonwoven web.

In the embodiment where the nonwoven web comprised by or forming the intermediate layer comprises first thermoplastic fibers having a first melting temperature and second thermoplastic fibers having a second melting temperature, a difference of the first melting temperature and the second melting temperature is at least about 40° C., the nonwoven web may comprise at least 30 wt %, or at least 40 wt %, or at least 50 wt % of the first or the second thermoplastic fibers whichever having a higher melting temperature based on the total weight of the nonwoven web.

In the embodiment where the nonwoven web comprised by or forming the intermediate layer comprises first thermoplastic fibers having a first melting temperature and second thermoplastic fibers having a second melting temperature, a difference of the first melting temperature and the second melting temperature is at least about 40° C., fibers having a lower melting temperature may be heat-fused one another, and/or substantial part of fibers having a higher melting temperature may not heat-fused one another.

In one embodiment when the second thermoplastic fibers have a melting temperature greater at least about 40° C. than the first thermoplastic fibers, the first thermoplastic fibers having a melting temperature lower than the second thermoplastic fibers, hollow fibers in this case, in the nonwoven web are heat-fused one another. The presence of the first thermoplastic fibers which are not heat-fused one another is acceptable as long as majority of the first thermoplastic fibers are heat-fused one another. The second thermoplastic fibers, hollow fibers in this case, in the nonwoven web are not heat-fused one another. Further, majorities of the first thermoplastic fibers and the second thermoplastic fibers may not be heat-fused each another.

Without being bound by theory, optimizing fiber to fiber bonding per mass of nonwoven web may enable the intermediate layer to have a high thickness under compression and a low stiffness especially in the crotch. Increase of fiber to fiber bonding in the nonwoven may increase the stiffness of the material. On the other hand, decrease of fiber-to-fiber bonding in nonwoven web may result in less integrity of the nonwoven which is more prone to collapse of the material under compressive forces.

The first thermoplastic fiber may be a solid round fiber, a hollow fiber or a shaped fiber. The second thermoplastic fiber may be a solid round fiber, a hollow fiber or a shaped fiber. In one embodiment, the second thermoplastic fiber is a hollow fiber or a shaped fiber. In the embodiment, the second thermoplastic fiber may be hollow conjugate fiber.

Shaped fibers also may introduce higher specific surface area which increases the capillary pressure of the second web layer containing shaped fibers which can lead to better drainage of the first web layer by the second fiber web layer comprising shape fibers. Shaped fibers may include bilobal shaped, trilobal shaped, quatro-lobal shaped, delta shaped, concave delta shaped, crescent shaped, oval shaped, star shaped, square shaped, U-shaped, H-shaped, C-shaped, V-shaped, diamond shaped fibers.

Hollow fibers enable greater loft with larger effective diameter per linear density with less weight. They also provide better resilience under compression. Hallow fibers can be hollow conjugate fibers with spiral and/or 3D crimp to maximize the benefits of loft and resilience. Such hollow conjugate fibers can have non-uniform properties across the fiber cross-section for instance by using polymers with different characteristics (e.g. different polymers or same polymer with different characteristics such as viscosity).

Without being bound by theory, hollow fibers or shaped fibers may be advantageous over solid round fibers to provide improved cushiony characteristics as hollow fibers and shaped fibers have higher resilience at the same fiber denier due to having higher effective radius compared to round fibers.

Each of the first and the second thermoplastic fibers may be monocomponent fibers or multicomponent fibers, such as bicomponent fibers. If the fibers are bicomponent fibers, they have a core-sheath configuration, wherein the core component has a higher melting temperature than the sheath component.

The intermediate layer comprises or consists of a nonwoven web which is air-through bonded. Such nonwoven webs generally have high loft. Hence, they have a porous structure to provide void volume for absorbing and temporarily holding liquid. At the same time, they provide softness and do not have an excessively high bending stiffness.

The fibers may be continuous, such as in a spunlaid nonwoven web. The spunlaid nonwoven web is preferably air-through bonded or spunlace. In addition to hydroentanglement (spunlace) or air-through bonding, the spunlaid nonwoven web may or may not have undergone some localized bonding with heat and/or pressure (e.g. point bonding), introducing localized bond regions where the fibers are fused to each other.

In some embodiments, the fibers comprised by the intermediate layer are staple fibers. Similar to a nonwoven web made of continuous fibers, a nonwoven web of staple fibers is preferably carded nonwoven such as air-through bonding nonwoven. In addition to air-through bonding, the nonwoven web of staple fibers may or may not have undergone some localized bonding with heat and/or pressure (e.g. point bonding), introducing localized bond regions where the fibers are fused to each other.

Irrespective whether the nonwoven web is made of continuous fibers or staple fibers, the localized bonding should however not bond an excessively large surface area, thus negatively impacting the loft and void volume of the nonwoven web as well as stiffness. Preferably, the total bond area obtained by localized bonding with heat and/or pressure (in addition to hydroentanglement or air-through bonding) should not be more than 20%, or not be more than 15%, or not be more than 10% of the total surface area of the nonwoven web.

Alternatively, the nonwoven web comprised by the intermediate layer should not have undergone any bonding and consolidation in addition to the hydroentanglement (spunlace) or air-through bonding. Thereby, the advantageous properties of such nonwoven webs can be used to their optimum.

Alternatively, the nonwoven web comprised by the intermediate layer should not have undergone any bonding and consolidation in addition to the hydroentanglement (spunlace) or air-through bonding. Thereby, the advantageous properties of such nonwoven webs can be used to their optimum.

In a spunlace nonwoven web the fibers have been subjected to hydroentanglement to intermingle and intertwine the fibers with each other. Cohesion and the interlacing of the fibers with one another may be obtained by means of a plurality of jets of water under pressure passing through a moving fleece or cloth and, like needles, causing the fibers to intermingle with one another. Thus, consolidation of a spunlace nonwoven web is essentially a result of hydraulic interlacing. “Spunlace nonwoven web”, as used herein, also relates to a nonwoven formed of two or more precursor webs, which are combined with each other by hydraulic interlacing. The two or more webs, prior to being combined into one nonwoven by hydraulic interlacing, may have underdone bonding processes, such as heat and/or pressure bonding by using e.g. a patterned calendar roll and an anvil roll to impart a bonding pattern. However, the two or more webs are combined with each other solely by hydraulic interlacing. Alternatively, the spunlace nonwoven web is a single web, i.e. it is not formed of two or more precursor webs. Spunlace nonwoven layers/webs can be made of staple fibers or continuous fibers.

Through-air bonding (interchangeably used with the term “air-through bonding”) means a process of bonding staple fibers or continuous fibers by forcing air through the nonwoven web, wherein the air is sufficiently hot to melt (or at least partly melt, or melt to a state where the fiber surface becomes sufficiently tacky) the polymer of a fiber or, if the fibers are multicomponent fibers, wherein the air is sufficiently hot to melt (or at least partly melt, or melt to a state where the fiber surface becomes sufficiently tacky) one of the polymers of which the fibers of the nonwoven web are made. The air velocity is typically between 30 and 90 meter per minute and the dwell time may be as long as 6 seconds. The melting and re-solidification of the polymer provide the bonding between different fibers.

### Other Components of the Central Chassis

Still referring to FIG. 5, the central chassis (38) may further comprise components that improve the fit of the article around the legs of the wearer, in particular barrier leg cuffs (31) and gasketing leg cuffs (34). The barrier leg cuffs (31) may be formed by a piece of material, typically a nonwoven, which is partially bonded to the rest of the article and can be partially raised away and thus stand up from the plane defined by the topsheet (24). The barrier leg cuffs (31) are typically delimited by a proximal edge joined to the rest of the article, typically the topsheet (24) and/or the backsheet (25), and a free terminal edge intended to contact and form a seal with the wearer's skin. The standing up portion of the cuffs typically comprise an elastic element, for example one or a plurality of elastic strands (35). The barrier leg cuffs (31) provide improved containment of liquids and other body exudates approximately at the junction of the torso and legs of the wearer.

In addition to the barrier leg cuffs (31), the article may comprise gasketing leg cuffs (34), which are formed in the same plane as the chassis of the central chassis (38), in particular which may be at least partially enclosed between the topsheet (24) or the barrier leg cuffs (31) and the backsheet (25), and may be placed laterally outwardly relative to the upstanding barrier leg cuffs (31). The gasketing leg cuffs (34) can provide a better seal around the thighs of the wearer. Usually each gasketing leg cuff (34) will comprise one or more elastic string or elastic element (33) comprised in the chassis of the diaper for example between the topsheet (24) and backsheet (25) in the area of the leg openings.

### Laminate Bonding

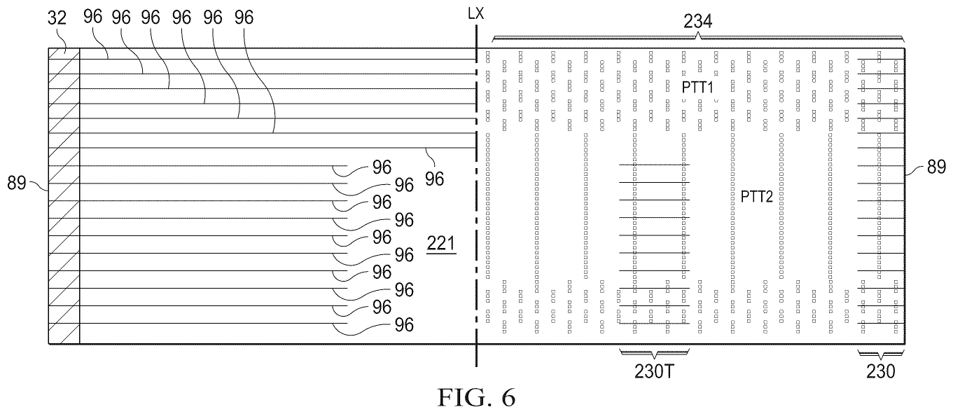

Referring to FIG. 2, the front belt (84) and the back belt (86) may each comprise a laminate, the laminate comprising a plurality of elastic members (96) running in the transverse direction, an inner sheet (94), an outer sheet (92), and an outer sheet fold over (not shown) wherein the outer sheet fold over is an extension of the outer sheet material formed by folding the outer sheet material at the distal edge (88) of the front and back belts; wherein the belt elastic members (96) are sandwiched between two of these sheets. The longitudinal dimension between adjacent elastic members (96) form an elastic spacing. The front elastic belt (84) and the back elastic belt (86) may each be made only by elastic members (96), the inner sheet (94), the outer sheet (92), and the outer sheet fold over. The belt elastic members (96) may extend in the transverse direction to provide a ring like elastic belt (40) when the front elastic belt (84) and the back elastic belt (86) are joined. At least some of the elastic members (96) extend in the transverse direction substantially parallel to each other. All of the elastic members (96) may extend in the transverse direction substantially parallel to each other. Such an article may be economically made. The front and back elastic belt (84, 86) each may have transversely continuous proximal and distal edges, the proximal edge (90) being located closer than the distal edge (88) relative to the longitudinal center of the article. At least 10%, or at least from about 15% to not more than about 70%, of the front and back elastic belts from the waist opening in the longitudinal direction may be a laminate in active elasticity along the entire transverse dimension LW of the front and back elastic belts (84, 86). Referring to FIGS. 2 and 6, the front and back elastic belts (84, 86) may be treated such that certain regions are removed of its elastic activity to form a non-elastic region (221). For each front and back elastic belt (84, 86), the region overlapping the front and/or back waist panel (52, 54) of the central chassis (38) may be removed of its elastic activity and defining the non-elastic region (221).

The longitudinal length of the backsheet (25) and the outer cover layer (42) may be the same, or may be varied. For example, the outer cover layer (42) may have a shorter longitudinal dimension compared to that of the backsheet (25), such that the outer cover layer (42) is devoid where the central chassis (38) overlaps the elastic belt (40). By such configuration, the elastic belt may have better breathability. Further, such configuration may provide cost saving. The transverse width of the backsheet (25) and the outer cover layer (42) may be the same, or may be varied. For example, the backsheet (25) may have a shorter transverse width compared to that of the outer cover layer (42). By such configuration, the longitudinal side edges (48) of the crotch panel (56), which make part of the leg openings, may have better breathability. Further, such configuration may provide cost saving.

As mentioned above, the elastic belt region (40) may be closely associated with the function and quality of the article. Thus, the gathering profile of the elastic belt region is also carefully selected by the manufacturer to provide the desired tactile and visible senses. Tactile sense such as flexibility and cushiony touch may enhance perception of high quality. The appearance of gathers may intuitively connote the function of the article, or the function of the elastic belt region (40). For example, relatively big uniform gathers may connote a fluffy and soft feel. For example, a bubble kind of texture may connote a soft and cushiony feel. Further, other functions provided by the laminate such as stretchability for ease of application, comfort and softness, as well as breathability, may enhance the perception provided by the gather appearance. Gathers intentionally provided to have a certain appearance may intuitively communicate the functional benefits described above, and provide the favorable entire usage experience of the article by the user. The user may be the wearer or the caregiver.

The laminate having improved function intuitive appearance of the present invention may be made by selecting the material for making the laminate, and by bonding the elastic members (96) and inner/outer sheets (92, 94) in a certain arrangement. The laminate may be made by bonding the elastic members (96) to at least one of the inner sheet (94) and the outer sheet (92), by an elastic bonding (230), and bonding the inner and outer sheets (92, 94) by a sheet bonding. The laminate may be made by bonding the elastic members (96) to at least one of the inner sheet (94) and the outer sheet (92), via a combination of an elastic bonding (230) and a sheet bonding. The sheet material for providing the laminate may be selected as described above, and to have a difference in basis weight of the inner sheet (94) and the outer sheet (92). Further, the laminate may be made by bonding the elastic members (96) in an appropriate denier, longitudinal pitch, and force; to one or both of the inner sheet (94) and the outer sheet (92).

What is meant by elastic bonding (230) herein is a bonding that bonds the elastic member (96) along the side edges (89) of the front and back elastic belts (84, 86). Such elastic bonding (230) may be provided by adhesive, heating, or ultrasound. The elastic bonding (230) may be continuously applied to each elastic member (96) for a length of at least about 10 mm, or from about 10 mm to about (60) mm in the direction of stretch adjacent the side edges (89) of the front and back elastic belts (84, 86), including the length planned for side seaming. The elastic bonding (230) is to provide relatively strong bonding for the elastic member (96) and thus securely anchor the elastic member (96) within the laminate. The anchoring may be assisted by the side seaming. A certain percentage, or a greater percentage, of the dimension of the elastic bonding (230) along the side edges (89) may be seamed. The elastic bonding (230) may also be utilized for an effective process of deactivating a limited transverse dimension of the elastic member (96). Referring to FIGS. 2 and 6, the elastic member (96) may be deactivated in portions overlapping the absorbent core (62). In addition to the side edge regions, the elastic bonding (230T) may be provided on both sides of the certain transverse dimension of the elastic member (96) which is planned to be deactivated, wherein the portion of the elastic member between the elastic bondings (230T) are severed and deactivated. The deactivated portions of the elastic member is not shown in FIGS. 2 and 6. Such deactivation may be referred to herein as tummy cut, and the deactivated region may match the non-elastic region (221).

What is meant by a sheet bonding herein is a bonding applied to at least one of the inner sheet (94) and the outer sheet (92) for bonding the inner sheet (94) and the outer sheet (92). The sheet bonding may be provided in the spacing of the elastic members and extend in the transverse direction. The sheet bonding may be provided to extend in the longitudinal direction, and thus cross the elastic members. The sheet bonding may be provided in discrete bond units (234) having a certain longitudinal and transverse direction, repeated for a specific area, or the entire area of the inner sheet (94) or the outer sheet (92) to be bonded with one another.

The sheet bonding may be a plurality of discrete bond units (234) herein is a bonding applied to at least one of the inner sheet (94) and the outer sheet (92) for intermittently bonding the inner sheet (94) and the outer sheet (92). Such discrete bond unit (230) may be provided by adhesive, heating, or ultrasound. Each discrete bond unit may have a longitudinal dimension of from about 0.5 mm to about 20 mm, preferably from about 0.5 mm to about 6.0 mm, and a transverse dimension of from about 0.5 mm to about 6.0 mm, preferably from about 0.5 mm to about 2.0 mm, wherein between any two discrete bond units, the discrete bond units have a longitudinal spacing of at least about 0.2 mm with each other, and a transverse spacing of at least about 0.2 mm with each other. All of the discrete bond units may be provided in the same longitudinal dimension and the same transverse dimension, respectively. Discrete bond units having different longitudinal and/or transverse dimensions may be used. The shape of the bond may be rectangular, circular, or oval.

Referring to FIG. 6, the laminate may be made by bonding the elastic members (96) to at least one of the inner sheet (94) and the outer sheet (92), via a combination of an elastic bonding (230) and plurality of discrete bond units (234). In FIG. 6, the laminate is shown with the elastic members (96) and elastic bonding (230) expressed in solid lines. The plurality of discrete bond units (234) is only expressed in the right side of the front elastic belt (84), and the side seams (32) are shown in an unjoined state.

The plurality of discrete bond units (234) are disposed such that there is at least one discrete bond unit disposed in each elastic spacing. By at least one discrete bond unit disposed in each elastic spacing, what is meant is that a discrete bond unit (234) in its complete longitudinal and transverse dimensions exists in the elastic spacing without contacting the elastic (96). For example, referring to FIG. 6, at least 2 discrete bond units (234) exist in each elastic spacing. By providing at least one discrete bond unit in each elastic spacing, the elastic members (96) are prevented from contacting each other. In that the elastic bonding (230) provides secure bonding of the elastic member (96) along the side seams (32), as well as the outer periphery of the non-elastic region (221), so long as there is at least one discrete bond unit (234) disposed in each elastic spacing, this prevents the elastic member (96) from moving away from its intended position. The plurality of discrete bond units (234) may also bond the elastic member (96) to at least one of the inner sheet (94) and the outer sheet (92). For an entire front elastic belt (84) or an entire back elastic belt (86), there may be no elastic member (96) bonded to the inner sheet (94) or the outer sheet (92) by a discrete bond unit (234). For an entire front elastic belt (84) or an entire back elastic belt (86), at least one to about 80% of the elastic members (96) may be bonded to the inner sheet (94) or the outer sheet (92) by a discrete bond unit (234). The plurality of discrete bond units (234) may only be provided to the outer sheet (92). The plurality of discrete bond units (234) may only be provided to the inner sheet (94). Referring to FIG. 6, the plurality of discrete bond units (234) may be provided for the entire area of the laminate. By providing the plurality of discrete bond units (234) for the entire area of the laminate, the plurality of discrete bond units (234) may serve as a bonding for the inner and outer sheets (92, 94) in regions where the elastic members (96) are severed. The plurality of discrete bond units (234) may be provided in regions adjacent the side edges (89) and thus overlapping the regions where the elastic bondings (230) are provided. Alternatively, the plurality of discrete bond units (234) may be provided only in regions where the elastic bondings (230) are not provided. The plurality of discrete bond units (234) may be provided at least in regions where the elastic member (96) is in active elasticity, wherein the elastic bondings (230) are devoid.

As mentioned above, all of the discrete bond units (234) may be provided in the same longitudinal dimension and the same transverse dimension, respectively. By providing each discrete bond unit in such way, and in a small enough dimension, various patterns may be created by the collection of discrete bond units.

### Application Means

The wearable article of the present invention may be assembled together with a different kind of application means than an elastic belt. Referring to FIG. 1B, the wearable article may be the taped type wherein the application means is a fastening system comprising a pair of elongate members (190) and a receiving member (192), the elongate members (190) transversely protruding from the left and right side edges of the back region of the central chassis and fastenable with the receiving member (192) disposed on the front region. Alternatively, the elongate members (190) may be protruding from the front region and fastenable with the receiving member (192) on the back region. The elongate members (190) may comprise an attaching portion, an extending portion, and refastenable means. The extending portion may be made of highly stretchable laminate for receiving stretching force upon applying the wearable article, and the refastenable means may be made of material physically engageable with materials of the receiving member (192). The combination of materials useful for the refastenable means and the receiving member (192) include hook and loop, latch and hole, button and hole, hook and hole, low tackifying adhesive agent, and combinations thereof. The receiving member (192) may also have a protruding portion which may or may not be equipped with refastenable means.

### MEASUREMENT METHODS

### Basis Weight of Nonwoven Substrate

The basis weight of nonwoven substrates are measured according to “ISO 9073-1:1989 Textiles—Test methods for nonwovens—Part 1: Determination of mass per unit area”. To obtain the nonwoven sample, cut a rectangle-shaped nonwoven specimen from the article with an area of 100 cm² (for example, 100 mm×100 mm), and measure its basis weight following the measurement principle used by the standard method above. The reported basis weight will be the average value of at least five replicates is reported to the nearest 1 gsm (g/m²).

### Fiber Diameter and Weight Ratio of Multi-Fiber Layer Nonwoven

### (1) Nonwoven Sample Preparation and Conditioning

When available in its raw material form, a sample of a size of 5±1 cm×5±1 cm is cut from the raw material. Otherwise, the nonwoven sample in the form of an outer sheet (92) or an outer cover layer (42) is removed from a finished wearable article. For the purpose of removing the nonwoven from the finished article, a razor blade is used to excise a sample of a size of 5±1 cm×5±1 cm area from the underling layers of the article around the outer perimeter. (If the nonwoven is of insufficient size to permit a 5±1 cm×5±1 cm area to be excised from the wearable article, the largest square of nonwoven that can be extracted is excised and used as the sample henceforth.) As necessary, cryogenic spray (such as Cyto-Freeze, Control Company, Houston TX) can be used to remove the nonwoven from the underling layer. The nonwoven samples obtained from a finished wearable article are pre-conditioned at ambient temperature and relative humidity (about 23° C. and 50% RH respectively) for at least 4 hours before measurements.

### (2) Measurement of Fiber Diameter

To prepare the specimen for cross-section imaging, submerge the nonwoven sample obtained or removed from the wearable article in liquid nitrogen and use a razor blade to cut a 10 mm×4 mm specimen from the nonwoven. The specimen is mounted vertically on a specimen stage with the wearer facing side attached onto the specimen stage using carbon tape. The cross-sectional edge of the specimen is facing upwards and oriented such that it is substantially aligned to the horizontal direction for subsequent imaging. The specimen is sputtered with platinum to avoid electric charging and improve overall conductivity, under the conditions of 30 mA current and 120 second coating time.

Cross section images of specimen are taken using a Scanning Electron Microscope (SEM) such as Tabletop Microscope TM3000 (Hitachi, Japan), or equivalent. The platinum-coated specimen is subsequently transferred into the SEM specimen vacuum chamber for the imaging analysis. An appropriate magnification and working distance are chosen so that the cross-section specimen is suitably enlarged for fiber diameter measurement and imaged under an acceleration voltage of 5 kV. The specimen images are saved as 8-bit jpeg images containing a linear distance scale for calibration. Measurement of the fiber diameter is performed using an image analysis program such as ImageJ software (version 1.52p or above, National Institutes of Health, USA) or equivalent. Record the values of fiber diameter to the nearest 0.1 micron (as shown in FIGS. 3A-3C). Measure at least 10 replicates and report the average value to the precision described above.

### (3) Measurement of Weight Ratio

### (3-1) Instrument Description

3D x-ray specimen imaging is obtained on a micro-CT instrument (a suitable instrument is the Scanco μCT 100 available from Scanco Medical AG, Switzerland, or equivalent). The micro-CT instrument is a cone beam microtomograph with a shielded cabinet. A maintenance free x-ray tube is used as the source with an adjustable diameter focal spot. The x-ray beam passes through the specimen, where some of the x-rays are attenuated by the specimen. The extent of attenuation correlates to the mass of material the x-rays have to pass through. The transmitted x-rays continue on to the digital detector array and generate a 2D projection image of the specimen. A 3D image of the specimen is generated by collecting several individual projection images of the specimen as it is rotated, which are then reconstructed into a single 3D image. The instrument is interfaced with a computer running software to control the image acquisition and save the raw data.

### (3-2) Image Acquisition

To prepare the specimen for 3D x-ray imaging, the nonwoven sample obtained or removed from the wearable article is die cut to obtain a circular piece with a diameter of 8 mm. Record the mass of the cut piece. Multiple specimens taken from the same specimen can be analyzed and compared to each other. Avoid folds, wrinkles or tears when selecting a location for sampling. Care should be taken to minimize distortion and compression of the specimen while cutting. Set up and calibrate the micro-CT instrument according to the manufacturer's specifications. Place the specimen into an appropriate holder and support the specimen with a ring of low-density material. This will allow the central portion of the specimen to lay horizontal and be scanned without having any other materials directly adjacent to its upper and lower surfaces. Measurements should be taken in this region. The 3D image field of view is approximately 10 mm on each side in the xy-plane with a resolution of approximately 5600 by 5600 pixels, and with a sufficient number of 1.8 microns thick slices collected to fully include the z-direction of the specimen. The reconstructed 3D image contains isotropic voxels of 1.8 microns. Images were acquired with the source at 70 kVp and 57 μA with a 0.1 mm aluminum filter. A total of 3400 projections images were obtained with an integration time of 800 ms and 4 averages. The projection images are reconstructed into the 3D image and saved in 16-bit format to preserve the full detector output signal for analysis. For each μCT instrument, these current and voltage settings should be optimized to produce the maximum contrast in the projection data with sufficient x-ray penetration through the specimen, but once optimized held constant for all substantially similar specimens.

### (3-3) Image Processing Method

A square section of the nonwoven is cropped to approximately 3.2 mm×3.2 mm from the μCT voxel data. The analysis is performed on the 8-bit dataset that completely intersects the minimum X border, the maximum X border, the minimum Y border and the maximum Y border. A small non-material buffer region will exist between the minimum Z border and the maximum Z border. This region will consist of air or packing material. A Material Threshold is determined for the specimen by Otsu's method using Matlab's built in “multithresh” function. This threshold should identify the nonwoven material while nearly eliminating noise and packing material. Stray packing material should be removed from the data manually when necessary.

Applying the Material Threshold results in a binary dataset with the fiber voxels labeled as “1”. The approximate thickness of the fibers can be accurately estimated with a Local Thickness Map (LTM). A LTM dataset is the same size as the fiber dataset. Every fiber voxel location in the LTM is assigned the radius value of the largest sphere within the fiber's morphology that encloses that fiber voxel. Finding and using local thickness values was first discussed in:

Hildebrand T, Rüegsegger P (1997) A new method for the model-independent assessment of thickness in three-dimensional images. J. Microsc. 185: 67-75.

A LTM algorithm was implemented in Matlab and executed on the fiber labelled dataset. The bisecting profile of the fibers is nearly circular making the radius of the maximum enclosing sphere a good estimation of the radius of the fiber at that voxel location. A Radius Threshold which separates the smaller diameter fiber voxels from the larger diameter fiber voxels is determined for the dataset by passing all fiber voxels to Matlab's “multithresh” function. LTM values greater than the Radius Threshold will result in a binary mask where nearly all the large diameter fiber voxels are labeled as “1”.

μCT scanning is an x-ray technology where intensity values are proportional to the density of the fiber material. A voxel is a cubic volume in space. The intensity of each fiber voxel therefore is proportional to the weight of the fiber within that cubic volume. Basis Weight is defined as weight over area. Therefore, summing all fiber voxel values and dividing that summation over the X-Y footprint (3.2 mm×3.2 mm) will give a value that is proportional to the Basis Weight of the specimen. The large diameter fiber ratio of that Basis Weight can be defined according to the following formula:

$$Large Fiber Ratio Of Basis Weight = \frac{\sum Large Fiber Voxel Intensities}{\sum All Fiber Voxel Intensities}$$

The small diameter fiber percentage of that basis weight can be defined as below and reported to the nearest 0.1%:

$$Small Fiber Of Basis Weight \% = 100 \% \times ( 1 - Large Fiber Ratio Of Basis Weight )$$

Repeat the measurement and analysis on multiple specimens. The arithmetic mean of the “Small Fiber Of Basis Weight” from at least three replicate specimens will be reported as the weight ratio (%) of the garment facing layer to the nearest 0.1%.

### Thickness Under Compression

### (1) Sample Preparation

To obtain a sample from the crotch region (30) of the finished wearable article, remove any cuffs and deactivate any elastic portion on both sides so that the remaining article can be placed flat on a bench. For a pants-type article, tear open the elastic belt along the side seam in advance. A square-shaped sample with the size of 110 mm×110 mm is cut out from the front side of the article using a paper trimmer, which has one side centrally located along the lateral centerline of the article and extends towards the elastic belt. The sample needs to be pre-conditioned in a room maintained at 23±2° C. and 50±5% relative humidity, for at least 4 hours prior to testing.

### (2) Thickness Under Compression

Thickness Under Compression of the samples are measured using the Fabric Touch Tester (FTT® M293) and FTT® system software available from SDL Atlas, or equivalent. FTT® system includes five modules (i.e., compression, bending, surface friction, roughness, and thermal properties) that can be activated at the same time for recording the dynamic responses from the samples if needed. The measurement for Thickness Under Compression only requires the compression module. The instrument is calibrated according to the manufacturer's instructions, using the standard calibration fabrics provided along with the instrument. All the testing is performed in a room maintained at 23±2° C. and 50±5% relative humidity. The test procedures are conducted according to the operating instructions given in the FTT M293 manual.

The 110 mm×110 mm sample with garment facing side upward is placed centrally on the lower plate in the FTT® system. The compression measurement is undertaken with single surface testing mode, when the sample is pushed downwards by the upper plate in the FTT® system that applies a continuously increasing normal force from 0 to 8470 gf (i.e. 0 to 70 gf/cm² in pressure).

Compression Work (CW) denotes the total work done on the sample during the compression process. Integral of the compression curve according to equation (1) is calculated to give the value of Compression Work in the unit of gf×mm, wherein Dₐ is the initial sample thickness at zero pressure, D_{c} is minimum sample thickness at maximum pressure, F is the measured force and D is the measured thickness during compression. The reported values will be the arithmetic mean of five replicate samples to the nearest 1 gf×mm.

$$C W = {\int }_{{D}_{a}}^{{D}_{c}} FdD \qquad ( 1 )$$

Thickness Under Compression of the sample is the measured thickness under the pressure of 41 gf/cm², during the compression test. The reported values will be the arithmetic mean of five replicate samples to the nearest 0.01 mm.

### Stretch Circumference Force and Fit Circumference Force

Force is measured using an Electronic Tensile Tester with a computer interface such as the MTS Criterion C42 running TestWorks 4 Software (available from MTS SYSTEMS (CHINA) CO., LTD) or equivalent instrument. A load cell is selected so that force results for the samples tested will be between 10 and 90% of capacity of the load cell used. The instrument is calibrated according to the manufacturer's instructions. All testing is performed in a room maintained at 23±2° C. and 50±5% relative humidity.

The tensile tester is fitted with hanger-type sample holding fixtures (300) as shown in FIG. 4. Each fixture comprises a rigid linear rubber-coated horizontal bar section (302) to prevent sample slippage during testing. The outer bar diameter (including the rubber coating) of the horizontal bar sections is 10.0 mm. The central axes of the horizontal bar sections (302) are configured to remain parallel and in the same vertical plane throughout the test procedure. The gauge circumference is determined by the following equation:

$$Gauge Circumference = 2 \times ( H + D + π D / 2 )$$

where H is the vertical gap between the horizontal bar sections (302), and D is the outer diameter of the bar.

The instrument is set up to go through the following steps:

| Crosshead Speed | 254.0 | mm/min |
|---|---|---|
| Final Load Point | 19.61N | |
| Hold Time | 0 | |
| Number of Cycles | 1 | |
| Data Acquisition Rate | 50 | Hz |

An article (20) sample is inserted onto the upper horizontal bar section (302) so that the bar passes through the waist opening and one leg opening of the article. The crosshead is raised until the specimen hangs above the lower bar and does not touch lower bar (302). The load cell is tared and the crosshead is lowered to enable the lower bar (302) to be inserted through the waist opening and other leg opening without stretching the article. The article is adjusted so that the longitudinal centerline LX of the article is in a horizontal plane halfway between the upper and lower bars (302). The center of the side portion in contact with the bar (302) is situated on the same vertical axis as the instrument load cell. The crosshead is raised slowly while the article is held in place by hand as necessary until the force is between 0.05 and 0.1N, while taking care not to add any unnecessary force. The gauge circumference at this point is the Initial Gauge Circumference. The test is initiated and the crosshead moves up at 254 mm/min until a force of 19.6N is attained, then the crosshead immediately returns to the Initial Gauge Circumference at the same speed. The maximum circumference at 19.6N and the force at 70% of the maximum circumference during the loading segment and unloading segment of the test are recorded.

The maximum circumference (mm) at 19.6N is defined as the Full Stretch Circumference W1. The Full Stretch Circumference (mm)×0.7 is defined as the 70% Stretch Circumference W2. The force (N) during the loading segment of the test at 70% Stretch Circumference is defined as the Stretch Circumference Force. The force (N) during the unloading segment of the test at 70% Stretch Circumference is defined as the Fit Circumference Force. Five samples are analyzed and their average are calculated and reported to the nearest 1 mm or 0.01N, respectively.

### Belt Minimum Peel Strength, Belt Maximum Peel Strength, Top Bottom Difference, and Material Breaking Point

### 1. Preparation of Finished Product Specimen

Specimen for the measurements hereinbelow are obtained from a finished wearable article sample, or ring-like elastic belt (104) sample, unless otherwise specified. To obtain a belt specimen from a finished wearable article sample, the belt is detached from the chassis (102) by hand.

Specimen are obtained from 6 (six) finished wearable articles from the same area of each article for each set of measurement. Specimen are pre-conditioned in a room maintained at 23±2° C. and 50±5% relative humidity, for at least 2 hours prior to testing. All testing is performed in a room maintained at 23±2° C. and 50±5% relative humidity.

### 2. Belt Minimum Peel Strength, Belt Maximum Peel Strength, Top Bottom Difference

As equipment, MTS Criterion C42 running TestWorks 4 Software with standard tensiometer jaw or equivalent is used.

4 (four) unique seam specimen are obtained from one belt specimen by cutting off the top (distal) edges and bottom (proximal) edges of the opposed longitudinal edges of the left and right seams in a longitudinal dimension (cross machine direction) of 25 mm and a lateral dimension (machine direction) of 50 mm by scissors. Care is taken such that, when an edge of the seam is discontinuous, such discontinuous portion is avoided and the continuous portion of the seam is sampled. Each of the 4 unique seam specimen from one belt specimen are provided identifiable as “top left”, “top right”, “bottom left”, and “bottom right”.
(1) The seam specimen is set such that the lateral direction of the belt matches the vertical direction of the equipment. The seam specimen is clamped between the upper and lower tensiometer jaws as straight as possible without applying pretension.
(2) The elongation measurement is taken from the point where the force curve leaves the zero line.
(3) A constant rate of extension of 460 mm/min is applied.
(4) Pull the seam specimen until the seam is completely separated. Record the peak force (N/25 mm).
(5) For each of the “top left”, “top right”, “bottom left”, and “bottom right” specimen, the average peak force of the 6 values of the 6 seam specimen are obtained and averaged, respectively, wherein each averaged value is named FTL, FTR, FBL, and FBR. The smallest of FTL, FTR, FBL, and FBR is the Belt Minimum Peel Strength, and the greatest of FTL, FTR, FBL, and FBR is the Belt Maximum Peel Strength.
(6) The Top Bottom Difference is obtained as such:

$$\vert \{ ( FTL + FTR ) - ( FBL + FBR ) \} \div ( FBL + FBR ) \vert ( \% )$$

### 3. Material Breaking Point

As equipment, MTS Criterion C42 running TestWorks 4 Software with standard tensiometer jaw or equivalent is used. For this measurement, raw material of the first substrate layer (162) and the raw material of the second substrate layer (164) is used. According to how the substrate is planned to be assembled, the substrate is cut in a lateral dimension (machine direction) of 25 mm and longitudinal dimension (cross machine direction) of 50 mm to provide a specimen. Thirty (30) specimens are obtained using different lots of substrate layers, or different regions of substrate layers.
(1) The specimen is set such that the longitudinal direction in which the layers are planned to be introduced to the belt matches the vertical direction of the equipment. The layers are clamped to leave 25 mm initial gauge length. The specimen is clamped between the upper and lower tensiometer jaws as straight as possible without applying pretension.
(2) A constant rate of extension of 2000 mm/min is applied.
(3) Pull the specimen until the specimen is completely broken. The force distribution up to 0.01N preciseness is recorded.
(4) The force (N) of highest frequency 1; is the Material Breaking Point (N).

### MD Tensile Strength/Basis Weight

MD tensile strength of a specimen is measured according to NWSP 110.4-09 with conditions below.
Test Speed: 100 mm/min
Sample Width: 50 mm
Sample length: sufficiently longer than gauge length
Gauge Length: 100 mm

### Thickness/Basis Weight

The Compression Average Rigidity (CAR), Standard Thickness (T), and Bending Work (BW) values are measured on a nonwoven test sample using a Fabric Touch Tester M293 (FTT), available from SDL Atlas USA, Rock Hill, SC, interfaced with a computer running FTT system software. According to SDL Atlas, the FTT objectively and quantitatively characterizes skin touch comfort by measuring various mechanical and surface properties. The FTT instrument offers a variety of assessment modules to measure these properties. The FTT Test utilizes the Compression Module, which compresses a sample between two plates while recording the applied normal force and corresponding distance between the plates during a compression and recovery cycle. The FTT Test also utilizes the Bending Module, which bends a sample over a bending bar while recording the bending force and corresponding bending angle. The recorded data is analyzed by the FTT software to calculate the CAR, T, and BW values. The instrument operation and testing procedures are performed according the instrument manufacture's specifications.

### 1 Sample Preparation

When a nonwoven is available in a raw material form, a rectangular test sample with a size of 310 mm×90 mm is cut from the raw material. When a nonwoven is a component of a finished product, the nonwoven is removed from the finished product using a razor blade to excise the nonwoven from other components of the finished product to provide a nonwoven test sample with a size of 310 mm×110 mm. A cryogenic spray (such as Cyto-Freeze, Control Company, Houston TX) may be used to remove the nonwoven specimen from other components of the finished product, if necessary. Equilibrate all samples at TAPPI standard temperature and relative humidity conditions (23° C.±2 C° and 50%±2%) for at least 4 hours prior to conducting the FTT testing, which is also conducted under TAPPI conditions.

### 2 Testing Procedure

The FTT instrument is calibrated according to the manufacturer's instructions using the provided standard calibration fabric. The test sample is placed into the instrument according to the manufacturer's instructions, with the appropriate amount of the sample laying on the compression plate and the remaining portion resting on the adjacent bending platform. The test sample should be laying flat and tension free prior to initiating the test. The compression and bending tests are initiated and performed according to the manufacturer's instructions.

When testing is complete, the FTT software displays values for CAR, T, and BW. Record each of these values. The test piece is then removed from the instrument and discarded. This testing procedure is performed individually on the other four replicate test samples.

The arithmetic means of the five recorded test result values for CAR, T, and BW are calculated and reported. Report the individual average values of CAR to the nearest 1 gf/mm³, T to the nearest 0.01 mm, and BW to the nearest 1 gf·mm·rad.

### Example

### Nonwoven Examples

A multi-fiber layer nonwoven of the present invention with tradename N_HB2008 available from Wisdom Nonwoven Co. LTD having Lot No. WSFS3C210712 was subjected to tests herein for obtaining the Fiber Diameter. Such multi-fiber layer nonwoven had 2 distinctive layers of fibers, and the fiber diameter of those layers were obtained according to measurements herein. The SEM photos of FIGS. 3A-3C were taken during the process of this measurement. Results are provided in Table 1A. Two types of such multi-fiber layer nonwoven, Nonwoven Example 1 having a basis weight of 20, and Nonwoven Example 2 having a basis weight of 25, were used in the Article Examples below. The weight ratio (%) of the garment facing layers were obtained according to measurements herein, and provided in Table 1B.

TABLE 1A

| Layer | Fiber Diameter (μm) |
|---|---|
| Garment facing | 10.4 |
| Wearer facing | 16.2 |

TABLE 1B

| Example | Weight ratio of garment-facing layer (%) |
|---|---|
| Nonwoven 1 | 51.0 |
| Nonwoven 2 | 40.3 |

### Article Examples

Article Examples 1-2 and Comparative Examples 1-3 were obtained as such and subject to the tests described below.

Example 1: Size 4 (L-size) belt-type pant article (Lot No. 20210828) having the Nonwoven Example 1 form the outer sheet and Nonwoven Example 2 form the outer cover layer, and having the configuration, elastic bonding, and pattern of discrete bond units of FIG. 6, elastic profile and other properties of Table 2 below, and including the intermediate layer in the central chassis.

Example 2: Size 4 (L-size) belt-type pant article (Lot No. 20210226) having the Nonwoven Example 2 form the outer cover layer, and having the configuration, elastic bonding, and pattern of discrete bond units of FIG. 6, and elastic profile and other properties of Table 2 below, and including the intermediate layer in the central chassis.

Comparative Example 1: “Ichiban Pants” Size 4 (Lot No. 20210223) purchased in February 2021 in PRC having the outer sheet and outer cover layer formed by a nonwoven similar to Nonwoven Example 1, however not having fibers with diameter less than 12 μm.

Comparative Example 2: “Huggies Penguin” Size 4 purchased in PRC having the outer sheet and outer cover layer formed by a nonwoven similar to Nonwoven Example 1, however not having fibers with diameter less than 12 μm. Lot No. 20210225 purchased in June 2021 was used for the measurement to obtain “Thickness Under Compression”, and Lot No. 20210412 purchased in July 2021 was used for the other tests.

Comparative Example 3: “Baby Care Royal Weak Acid” Size 4 purchased in PRC having the outer sheet and outer cover layer formed by a nonwoven similar to Nonwoven Example 1, however not having fibers with diameter less than 12 μm. Lot No. 20200902 purchased in June 2021 was used for the measurement to obtain “Thickness Under Compression”, and Lot No. #20210402 purchased in July 2021 was used for the other tests.

TABLE 2

| | Examples 1-2 |
|---|---|
| Zone | Dtex/elongation/Number of elastics |
| Front waist zone | 470Dtex/180%/7 |
| Front tummy zone | 940Dtex/190%/8 with tummy cut (*1) |
| Front leg zone | 470Dtex/180%/2 with tummy cut (*1) |
| Back waist zone | 470Dtex/180%/4 |
| | 940Dtex/130%/3 |
| Back tummy zone | 940Dtex/130%/1 |
| | 470Dtex/180%/4 |
| | 470Dtex/240%/1 with tummy cut (*1) |
| Back leg zone | 470Dtex/240%/2 with tummy cut (*1) |

(*1) “Tummy cut” in Table 2 refers to deactivation of elasticity at the transverse central area of elastic strands resulting in 68% effective length of elasticity.

### 1. Technical Measurements

The Thickness Under Compression was measured according to methods herein and results provided in Table 3 below.

TABLE 3

| | Exam- | Comparative | Comparative | Comparative |
|---|---|---|---|---|
| | ple 1 | Example 1 | Example 2 | Example 3 |
| Thickness Under | 2.85 | 2.35 | 3.44 | 3.60 |
| Compression (mm) | | | | |

### 2. Consumer Acceptance Test 1

60 panelists who were caregivers of babies using Size 4 (L size) pant diapers and having a mixture of usage experience of major brands of similar price range used in the test were recruited. There were about equal number of caregivers of boy and girl babies and having a weight of 9-14 kg. Five (5) finished product test samples (including Example 1 and Comparative Examples 2-3) were provided to the panelists to touch and feel the center of the product with their hands one by one. Each respondent was asked to fill in a questionnaire individually after touching the test sample one by one. In the questionnaire, there were 4 values as found in Table 4, and each respondent was requested to sort and rate the test samples against those values using the ratings from 1 to 10, which were scored as such: ‘1=poor, 10=excellent’. The scores were averaged.

TABLE 4

| | | Comparative | Comparative |
|---|---|---|---|
| Values | Example 1 | Example 2 | Example 3 |
| Overall rating | 75(*2) | 58 | 53 |
| Overall softness | 83(*3) | 76 | 60 |
| Belt softness | 83(*2) | 75 | 72 |
| Crotch softness | 84(*2) | 70(*3) | 55 |

(*2)These scores were statistically significantly better against Comparative Examples 2-3 at 90% confidence level.
(*3)These scores were statistically significantly better against Comparative Example 3 at 90% confidence level.

According to the test result in Table 4, Example 1 which meets the requirements of the present invention has statistically significantly higher values compared to the Comparative Examples.

### 2. Consumer Acceptance Test 2

107 panelists who were caregivers of babies using Size 4 (L size) pant diapers and having diaper usage of at least 3 pads/day in the past 7 day were recruited. There were about equal number of caregivers of boy and girl babies and having a weight of 9-14 kg.

Each panelist were provided enough test products of Example 1 and Comparative Example 1 for 4 consecutive days usage each. Panelists were asked to use only the test products, while following their normal usage frequency/habits. At the very beginning, the panelists were asked to take 1 pad to touch and feel, and asked to fill in the “Before usage” questions as in Table 5 by rating from 1 to 10, which were scored as such: ‘1=poor, 10=excellent’. After the 4 day usage the panelists were asked to repeat the same. The scores were averaged and provided in Table 5.

TABLE 5

| | | Comparative |
|---|---|---|
| Values | Example 2 | Example 1 |
| Before usage - Overall rating | 67(*4) | 59 |
| Before usage - Overall softness | 72(*4) | 58 |
| Before usage - Crotch softness | 72(*4) | 60 |
| After usage - Overall rating | 65 | 59 |
| After usage - Overall softness | 81(*4) | 63 |
| After usage - Crotch softness | 77(*4) | 66 |

(*4)Statistically significantly better against Comparative Example 1 at 90% confidence level.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Further, every numerical range given throughout this specification includes every narrower numerical range that falls within such broader numerical range.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A wearable article continuous in a longitudinal direction and a transverse direction comprising a front elastic belt region, a back elastic belt region, a crotch region, and a pair of side seams which join the front elastic belt region and the back elastic belt region to form a waist opening and a pair of leg openings; the crotch region extending longitudinally between the front elastic belt region and the back elastic belt region;
wherein each of the front and back elastic belt region comprises a laminate comprising an inner sheet, an outer sheet, and a plurality of elastic members running in the transverse direction;
wherein the outer sheet comprises a garment facing surface and a wearer facing surface, the outer sheet formed by a multi-fiber layer nonwoven having a basis weight of from about 16 gsm to about 35 gsm and comprising a garment facing layer and a wearer facing layer, the garment facing layer comprising fibers having a diameter of about 7 μm to about 11 μm, and the wearer facing layer comprising fibers having a diameter of about 13 μm to about 24 μm, wherein a weight ratio of the garment facing layer is from about 20% to about 70% of the multi-fiber layer nonwoven.

2. The wearable article of claim 1, wherein the fibers of the multi-fiber layer nonwoven comprise air-through carded fibers, and wherein the layers of the multi-fiber layer nonwoven are bonded by hot air through bonding.

3. The wearable article of claim 1, wherein the inner sheet has a melting point of no more than about 165° C.

4. The wearable article of claim 1, comprising a central chassis comprising a liquid permeable topsheet, a liquid impermeable backsheet, and an absorbent core disposed between the topsheet and the backsheet, wherein the wearable article has a Thickness Under Compression of from about 2.7 mm to about 4.0 mm.

5. The wearable article of claim 4, wherein the central chassis comprises an outer cover layer for covering the garment-facing side of the backsheet, wherein the outer cover layer is formed by the multi-fiber layer nonwoven, and wherein the outer cover layer has a shorter longitudinal dimension compared to that of the backsheet.

6. The wearable article of claim 4, wherein the central chassis bridges the front elastic belt region and the back elastic belt region, and wherein the front elastic belt region and the back elastic belt region are separated with the crotch region.

7. The wearable article of claim 1, wherein the side seams have a Belt Minimum Peel Strength of at least about 6N/25 mm, a Belt Maximum Peel Strength of no more than about 18N/25 mm, and a Top Bottom Difference of no more than about 15%, according to the test methods herein.

8. The wearable article of claim 7, wherein the outer sheet has a Material Breaking Point of at least about 7N, according to the test method herein.

9. The wearable article of claim 1, wherein the laminate comprises:
an elastic bonding which continuously bonds the elastic members for at least about 10 mm in the direction of stretch in a region adjacent the side edges of the front and back elastic belt regions1 and
a sheet bonding which bonds the inner sheet and the outer sheet.

10. The wearable article of claim 9, wherein the sheet bonding is a plurality of discrete bond units disposed between the elastic bondings in the transverse direction, each discrete bond unit applied to at least one of the inner sheet and the outer sheet, wherein there is at least one discrete bond unit disposed in each spacing between the elastic members.

11. The wearable article of claim 10, wherein the discrete bond unit has a longitudinal dimension of from about 0.5 mm to about 20 mm, and a transverse dimension of from about 0.5 mm to about 6.0 mm.

12. The wearable article of claim 1, wherein the wearable article has a Stretch Circumference Force of no more than about 6.5N, and a Fit Circumference Force of at least about 2.0N, according to the test methods herein.

13. The wearable article of claim 5, wherein the absorbent core comprises a high loft material encompassing superabsorbent polymer particles.

14. The wearable article of claim 5, wherein the absorbent core comprises an absorbent layer having superabsorbent polymers disposed between first and second layers of nonwoven material immobilized by a fibrous layer of thermoplastic adhesive material.

15. The wearable article of claim 13, comprising an acquisition system between the topsheet and the absorbent core, wherein the acquisition system comprises viscose.

16. The wearable article of claim 13, comprising an intermediate layer disposed between the absorbent core and the backsheet;
wherein the intermediate layer has an MD Tensile/Basis Weight of no greater than about 0.75 N/5 cm/g/m², and a Thickness/Basis Weight of no less than about 0.078 mm/g/m², according to the test methods herein.

17. The wearable article of claim 16, wherein the intermediate layer comprises a first thermoplastic fiber having a first melting temperature and a second thermoplastic fiber having a second melting temperature, and wherein the difference between the first melting temperature and the second melting temperature is at least about 40° C.

18. The wearable article of claim 17, wherein the first thermoplastic fibers are heat-fused to one another, and wherein the second thermoplastic fibers are hollow fibers or shaped fibers which are not heat-fused to one another.

19. The wearable article of claim 16, wherein the intermediate layer comprises a nonwoven web selected from the group of an air-through bonded nonwoven made of staple fibers, carded calendar bonded nonwovens made of staple fibers, and combinations thereof, a spunlace nonwoven made of staple fibers, an air-through bonded nonwoven made of spunlaid fibers, and a spunlace nonwoven made of spunlaid fibers.

20. A wearable article continuous in a longitudinal direction and a transverse direction comprising a front elastic belt region, a back elastic belt region, a crotch region, and a pair of side seams which join the front elastic belt region and the back elastic belt region to form a waist opening and a pair of leg openings; the crotch region extending longitudinally between the front elastic belt region and the back elastic belt region;
wherein each of the front and back elastic belt region comprises a laminate comprising an inner sheet, an outer sheet, and a plurality of elastic members running in the transverse direction;
wherein the outer sheet has a basis weight of from about 16 gsm to about 35 gsm and comprises a garment facing surface and a wearer facing surface, the backsheet nonwoven formed by a multi-fiber layer nonwoven comprising a garment facing layer and a wearer facing layer, the garment facing layer comprising fibers having a diameter of about 11 μm or less, and the wearer facing layer comprising fibers having a diameter of about 13 μm or more; and
wherein the side seams have a Belt Minimum Peel Strength of at least about 6 N/25 mm, a Belt Maximum Peel Strength of no more than about 18 N/25 mm, and a Top Bottom Difference of no more than about 15%, according to the test methods herein.
